(19) United States
(12) Patent Application Publication
(10) Pub. No.: US 2024/0081722 A1
(43) Pub. Date: Mar. 14, 2024

(54) Technique of Identifying Dementia

(71) Applicant: HAII corp., Seoul (KR)

(72) Inventors: Ho Yung KIM, Seoul (KR); Geon Ha KIM, Seoul (KR); Bo Hee KIM, Seoul (KR); Dong Han KIM, Seoul (KR); Hye Bin HWANG, Incheon (KR); Chan Yeong PARK, Seoul (KR); Ji An CHOI, Seoul (KR); Bo Ri KIM, Seoul (KR)

(73) Assignee: HAII corp., Seoul (KR)

(21) Appl. No.: 18/282,990

(22) PCT Filed: Jul. 7, 2022
(86) PCT No.: PCT/KR2022/009843

§ 371 Date: Sep. 19, 2023

(30) Foreign Application Priority Data
Jan. 5, 2022 (KR) 10-2022-0001430

(51) Int. Cl. A61B 5/00; A61B 5/16

(52) U.S. Cl. CPC A61B 5/4088; A61B 5/163; A61B 5/7435; A61B 5/7475; A61B 2503/08

(57) ABSTRACT

A method of identifying dementia is disclosed that includes causing a user terminal to display an N-th screen including a plurality of objects. The user terminal may further display an N+1-th screen with the objects rearranged at positions on the N+1-th screen which are different from positions of the objects included in the N-th screen when an N-th selection input of selecting any one from among the objects included in the N-th screen is received. When an N+1-th selection input for selecting any one from among the objects included in the N+1-th screen is received, a third task of determining whether an answer of the N+1-th selection input is correct is performed based on whether the object selected from the N+1-th selection input is the same as at least one object selected from at least one previous selection input including the N-th selection input.

### TECHNICAL FIELD

The present disclosure relates to a technique of identifying dementia, and more particularly to a device for identifying dementia using digital biomarkers according to tests and a method thereof.

### BACKGROUND ART

Alzheimer's disease (AD), which is a brain disease caused by aging, causes progressive memory impairment, cognitive deficits, changes in individual personality, etc. In addition, dementia refers to a state of persistent and overall cognitive function decline that occurs when a person who has led a normal life suffers from damage to brain function due to various causes. Here, cognitive function refers to various intellectual abilities such as memory, language ability, temporal and spatial understanding ability, judgment ability, and abstract thinking ability. Each cognitive function is closely related to a specific part of the brain. The most common form of dementia is Alzheimer's disease.

Various methods have been proposed for diagnosing Alzheimer's disease, dementia, or mild cognitive impairment. For example, a method of diagnosing Alzheimer's disease or mild cognitive impairment using the expression level of miR-206 in the olfactory tissue, a method for diagnosing dementia using a biomarker that characteristically increases in blood, and the like are known.

However, since special equipment or tests necessary for biopsy are required so as to use miR-206 in the olfactory tissue, and blood from a patient should be collected by an invasive method so as to use biomarkers in blood, there is a disadvantage that the patient's rejection feeling is relatively large.

Therefore, there is an urgent need for development of a dementia diagnosis method where patients hardly feel rejection without a separate special equipment or examination.

### DISCLOSURE

### Technical Problem

The present disclosure has been made in view of the above problems, and it is one object of the present disclosure to provide an accurate dementia diagnosis method where patients hardly feel rejection.

It will be understood that technical problems of the present disclosure are not limited to the aforementioned problem and other technical problems not referred to herein will be clearly understood by those skilled in the art from the description below.

### Technical Solution

In accordance with an aspect of the present disclosure, the above and other objects can be accomplished by the provision of a method of identifying dementia by at least one processor of a device, the method including: performing a first task of causing a user terminal to display an N-th screen including a plurality of objects, wherein the N is a natural number equal to or greater than 1: performing a second task of causing the user terminal to display an N+1-th screen wherein the plurality of objects is rearranged at positions on the N+1-th screen which are different from positions of the plurality of objects included in the N-th screen when an N-th selection input of selecting any one from among the plurality of objects included in the N-th screen is received; and performing, when an N+1-th selection input for selecting any one from among the plurality of objects included in the N+1-th screen is received, a third task of determining whether an answer of the N+1-th selection input is correct based on whether the object selected from the N+1-th selection input is the same as at least one object selected from at least one previous selection input including the N-th selection input.

According to some embodiments of the present disclosure, the method may further include: when the second task and the third task are performed a preset number of times and M is added to the N when the second task is performed M more times, where M is a natural number equal to or greater than 1.

According to some embodiments of the present disclosure, the method may further include: acquiring gaze information based on an image including user's eyes acquired in association with performing the first task and the second task.

According to some embodiments of the present disclosure, the gaze information may include at least one of information about an order in which the user's gaze moves, information on whether the user's gaze is maintained on each of the plurality of objects, and information on a time where the user's gaze is maintained on each of the plurality of objects.

According to some embodiments of the present disclosure, the method may include inputting at least one of the gaze information, result data obtained by performing the third task, and information on a response time of the user into a dementia identification model to calculate a score value; and determining whether the user has dementia based on the score value.

According to some embodiments of the present disclosure, the result data may include at least one of information on the number of times determined to be a current answer through the third task among the preset number of times and information on the number of times determined to be an incorrect answer through the third task among the preset number of times, and the information on response time may include information on a time taken until the N-th selection input or the N+1-th selection input is received in a state in which the N-th screen or the N+1-th screen is displayed.

According to some embodiments of the present disclosure, the second task may include a sub-task of inactivating an additional selection input for the N-th screen when the N-th selection input is received, and the third task may include a sub-task of inactivating an additional selection input for the N+1-th screen when the N+1-th selection input is received.

According to some embodiments of the present disclosure, the third task may include: an operation of determining that an answer is correct when all of the at least one object selected through the at least one previous selection input differ from the object selected from the N+1-th selection input; or an operation of determining that an answer is correct when any one of the at least one object selected from the at least one previous selection input is the same as the object selected from the N+1-th selection input.

According to some embodiments of the present disclosure, the second task may include a sub-task of randomly changing positions of the plurality of objects included in the N-th screen to rearrange the plurality of objects included in the N+1-th screen.

In accordance with another aspect of the present disclosure, there is provided a computer program stored on a computer-readable storage medium, the computer program performs processes of identifying dementia when executed on at least one processor of a device, the processes including: performing a first task of causing a user terminal to display an N-th screen including a plurality of objects, wherein the N is a natural number equal to or greater than 1; performing a second task of causing the user terminal to display an N+1-th screen wherein the plurality of objects is rearranged at positions on the N+1-th screen which are different from positions of the plurality of objects included in the N-th screen when an N-th selection input of selecting any one from among the plurality of objects included in the N-th screen is received; and performing, when an N+1-th selection input for selecting any one from among the plurality of objects included in the N+1-th screen is received, a third task of determining whether an answer of the N+1-th selection input is correct based on whether the object selected from the N+1-th selection input is the same as at least one object selected from at least one previous selection input including the N-th selection input.

In accordance with yet another aspect of the present disclosure, there is provided a device for identifying dementia, the device includes: a storage configured to store at least one program command; and at least one processor configured to perform the at least one program command, wherein the at least one processor performs a first task of causing a user terminal to display an N-th screen including a plurality of objects, wherein the N is a natural number equal to or greater than 1; performs a second task of causing the user terminal to display an N+1-th screen wherein the plurality of objects is rearranged at positions on the N+1-th screen which are different from positions of the plurality of objects included in the N-th screen when an N-th selection input of selecting any one from among the plurality of objects included in the N-th screen is received; and performs, when an N+1-th selection input for selecting any one from among the plurality of objects included in the N+1-th screen is received, a third task of determining whether an answer of the N+1-th selection input is correct based on whether the object selected from the N+1-th selection input is the same as at least one object selected from at least one previous selection input including the N-th selection input.

It will be understood that technical solutions of the present disclosure are not limited to the aforementioned solutions and other technical solutions not referred to herein will be clearly understood by those skilled in the art from the description below.

### Advantageous Effects

The effect of a technique of identifying dementia according to the present disclosure is as follows.

According to some embodiments of the present disclosure, provided is an accurate dementia diagnosis method where patients hardly feel rejection.

It will be understood that effects obtained by the present disclosure are not limited to the aforementioned effect and other effects not referred to herein will be clearly understood by those skilled in the art from the description below.

### DESCRIPTION OF DRAWINGS

Various embodiments of the present disclosure are described with reference to the accompanying drawings. Here, like reference numbers are used to refer to like elements. In the following embodiments, numerous specific details are set forth so as to provide a thorough understanding of one or more embodiments for purposes of explanation. It will be apparent, however, that such embodiment(s) may be practiced without these specific details.

FIG. 1 is a schematic diagram for explaining a system for identifying dementia according to some embodiments of the present disclosure.

FIG. 2 is a diagram for explaining an embodiment of a method of acquiring input data for dementia identification by a device according to some embodiments of the present disclosure.

FIG. 3 is a diagram for explaining an embodiment of a screen displayed on a user terminal according to some embodiments of the present disclosure.

FIG. 4 is a flowchart for explaining an embodiment of a method of determining whether a user has dementia according to some embodiments of the present disclosure.

### BEST MODE

Hereinafter, various embodiments of an apparatus according to the present disclosure and a method of controlling the same will be described in detail with reference to the accompanying drawings. Regardless of the reference numerals, the same or similar components are assigned the same reference numerals, and overlapping descriptions thereof will be omitted.

Objectives and effects of the present disclosure, and technical configurations for achieving the objectives and the effects will become apparent with reference to embodiments described below in detail in conjunction with the accompanying drawings. In describing one or more embodiments of the present disclosure, a detailed description of known functions and configurations incorporated herein will be omitted when it may make the subject matter of the present disclosure unclear.

The terms used in the specification are defined in consideration of functions used in the present disclosure, and can be changed according to the intent or conventionally used methods of clients, operators, and users. The features of the present disclosure will be more clearly understood from the accompanying drawings and should not be limited by the accompanying drawings, and it is to be appreciated that all changes, equivalents, and substitutes that do not depart from the spirit and technical scope of the present disclosure are encompassed in the present disclosure.

The suffixes “module” and “unit” of elements herein are used for convenience of description and thus can be used interchangeably and do not have any distinguishable meanings or functions.

Terms including an ordinal number, such as first, second, etc., may be used to describe various elements, but the elements are not limited by the terms. The above terms are used only for the purpose of distinguishing one component from another component. Therefore, a first component mentioned below may be a second component within the spirit of the present description.

A singular expression includes a plural expression unless the context clearly dictates otherwise. That is, a singular expression in the present disclosure and in the claims should generally be construed to mean “one or more” unless specified otherwise or if it is not clear from the context to refer to a singular form.

The terms such as “include” or “comprise” may be construed to denote a certain characteristic, number, step, operation, constituent element, or a combination thereof, but may not be construed to exclude the existence of or a possibility of addition of one or more other characteristics, numbers, steps, operations, constituent elements, or combinations thereof.

The term “or” in the present disclosure should be understood as “or” in an implicit sense and not “or” in an exclusive sense. That is, unless otherwise specified or clear from context, “X employs A or B” is intended to mean one of natural implicit substitutions. That is, when X employs A; when X employs B; or when X employs both A and B, “X employs A or B” can be applied to any one of these cases. Furthermore, the term “and/or” as used in the present disclosure should be understood to refer to and encompass all possible combinations of one or more of listed related items.

As used in the present disclosure, the terms “information” and “data” may be used interchangeably.

Unless otherwise defined, all terms (including technical and scientific terms) used in the present disclosure may be used with meanings that can be commonly understood by those of ordinary skill in the technical field of the present disclosure. Also, terms defined in general used dictionary are not to be excessively interpreted unless specifically defined.

However, the present disclosure is not limited to embodiments disclosed below and may be implemented in various different forms. Some embodiments of the present disclosure are provided merely to fully inform those of ordinary skill in the technical field of the present disclosure of the scope of the present disclosure, and the present disclosure is only defined by the scope of the claims. Therefore, the definition should be made based on the content throughout the present disclosure.

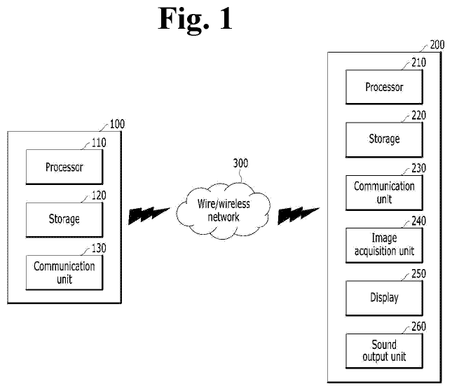

According to some embodiments of the present disclosure, at least one processor (hereinafter, referred to as a “processor”) of the device may determine whether a user has dementia using a dementia identification model. Specifically, the processor may acquire a score value by acquiring the user's gaze information acquired by performing tests; and information on test result data and a user's response time and then inputting the same into the dementia identification model. In addition, the processor may determine whether the user has dementia based on the score value. Hereinafter, a method of identifying dementia is described with reference to FIGS. 1 to 8.

FIG. 1 is a schematic diagram for explaining a system for identifying dementia according to some embodiments of the present disclosure.

Referring to FIG. 1, the system for identifying dementia may include a device 100 for identifying dementia and a user terminal 200 for a user requiring dementia identification. In addition, the device 100 and the user terminal 200 may be connected to communication through the wire/wireless network 300. However, the components constituting the system shown in FIG. 1 are not essential in implementing the system for identifying dementia, and thus more or fewer components than those listed above may be included.

The device 100 of the present disclosure may be paired with or connected to the user terminal 200 through the wire/wireless network 300, thereby transmitting/receiving predetermined data. have. In this case, data transmitted/received through the wire/wireless network 300 may be converted before transmission/reception. Here, the “wire/wireless network” 300 collectively refers to a communication network supporting various communication standards or protocols for pairing and/or data transmission/reception between the device 100 and the user terminal 200. The wire/wireless network 300 includes all communication networks to be supported now or in the future according to the standard and may support all of one or more communication protocols for the same.

The device 100 for identifying dementia may include a processor 110, a storage 120, and a communication unit 130. The components shown in FIG. 1 are not essential for implementing the device 100, and thus, the device 100 described in the present disclosure may include more or fewer components than those listed above.

Each component of the device 100 of the present disclosure may be integrated, added, or omitted according to the specifications of the device 100 that is actually implemented. That is, as needed, two or more components may be combined into one component or one component may be subdivided into two or more components. In addition, a function performed in each block is for explaining an embodiment of the present disclosure, and the specific operation or device does not limit the scope of the present disclosure.

The device 100 described in the present disclosure may include any device that transmits and receives at least one of data, content, service, and application, but the present disclosure is not limited thereto.

The device 100 of the present disclosure includes, for example, any standing devices such as a server, a personal computer (PC), a microprocessor, a mainframe computer, a digital processor and a device controller; and any mobile devices (or handheld device) such as a smart phone, a tablet PC, and a notebook, but the present disclosure is not limited thereto.

In the present disclosure, the term “server” refers to a device or system that supplies data to or receives data from various types of user terminals, i.e., a client. For example, a web server or portal server that provides a web page or a web content (or a web service), an advertising server that provides advertising data, a content server that provides content, an SNS server that provides a Social Network Service (SNS), a service server provided by a manufacturer, a Multichannel Video Programming Distributor (MVPD) that provides Video on Demand (VoD) or a streaming service, a service server that provides a pay service, or the like may be included as a server.

In the present disclosure, the device 100 means a server according to context, but may mean a fixed device or a mobile device, or may be used in an all-inclusive sense unless specified otherwise.

The processor 110 may generally control the overall operation of the device 100 in addition to an operation related to an application program. The processor 110 may provide or process appropriate information or functions by processing signals, data, information, etc. that are input or output through the components of the device 100 or driving an application program stored in the storage 120.

The processor 110 may control at least some of the components of the device 100 to drive an application program stored in the storage 120. Furthermore, the processor 110 may operate by combining at least two or more of the components included in the device 100 to drive the application program.

The processor 110 may include one or more cores, and may be any of a variety of commercial processors. For example, the processor 110 may include a Central Processing Unit (CPU), General Purpose Graphics Processing Unit (GPUGP), Tensor Processing Unit (TPU), and the like of the device. However, the present disclosure is not limited thereto.

The processor 110 of the present disclosure may be configured as a dual processor or other multiprocessor architecture. However, the present disclosure is not limited thereto.

The processor 110 may identify whether a user has dementia using the dementia identification model according to some embodiments of the present disclosure by reading a computer program stored in the storage 120.

The storage 120 may store data supporting various functions of the device 100. The storage 120 may store a plurality of application programs (or applications) driven in the device 100, and data, commands, and at least one program command for the operation of the device 100. At least some of these application programs may be downloaded from an external server through wireless communication. In addition, at least some of these application programs may exist in the device 100 from the time of shipment for basic functions of the device 100. Meanwhile, the application program may be stored in the storage 120, installed in the device 100, and driven by the processor 110 to perform the operation (or function) of the device 100.

The storage 120 may store any type of information generated or determined by the processor 110 and any type of information received through the communication unit 130.

The storage 120 may include at least one type of storage medium of a flash memory type, a hard disk type, a Solid State Disk (SSD) type, a Silicon Disk Drive (SDD) type, a multimedia card micro type, a card-type memory (e.g., SD memory, XD memory, etc.), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disk. The device 100 may be operated in relation to a web storage that performs a storage function of the storage 120 on the Internet.

The communication unit 130 may include one or more modules that enable wire/wireless communication between the device 100 and a wire/wireless communication system, between the device 100 and another device, or between the device 100 and an external server. In addition, the communication unit 130 may include one or more modules that connect the device 100 to one or more networks.

The communication unit 130 refers to a module for wired/wireless Internet connection, and may be built-in or external to the device 100. The communication unit 130 may be configured to transmit and receive wire/wireless signals.

The communication unit 130 may transmit/receive a radio signal with at least one of a base station, an external terminal, and a server on a mobile communication network constructed according to technical standards or communication methods for mobile communication (e.g., Global System for Mobile communication (GSM), Code Division Multi Access (CDMA), Code Division Multi Access 2000 (CDMA2000), Enhanced Voice-Data Optimized or Enhanced Voice-Data Only (EV-DO), Wideband CDMA (WCDMA), High Speed Downlink Packet Access (HSDPA), High Speed Uplink Packet Access (HSUPA), Long Term Evolution (LTE), Long Term Evolution-Advanced (LTE-A), etc.).

An example of wireless Internet technology includes Wireless LAN (WLAN), Wireless-Fidelity (Wi-Fi), Wireless Fidelity (Wi-Fi) Direct, Digital Living Network Alliance (DLNA), Wireless Broadband (WiBro), World Interoperability for Microwave Access (WiMAX), High Speed Downlink Packet Access (HSDPA), High Speed Uplink Packet Access (HSUPA), Long Term Evolution (LTE), Long Term Evolution-Advanced (LTE-A), and the like. However, in a range including Internet technologies not listed above, the communication unit 130 may transmit/receive data according to at least one wireless Internet technology.

In addition, the communication unit 130 may be configured to transmit and receive signals through short range communication. The communication unit 130 may perform short range communication using at least one of Bluetooth [symbol] , Radio Frequency Identification (RFID), Infrared Data Association (IrDA), Ultra-Wideband (UWB), ZigBee, Near Field Communication (NFC), Wireless-Fidelity (Wi-Fi), Wi-Fi Direct and Wireless Universal Serial Bus (Wireless USB) technology. The communication unit 130 may support wireless communication through short range communication networks (wireless area networks). The short range communication networks may be wireless personal area networks.

The device 100 according to some embodiments of the present disclosure may be connected to the user terminal 200 and the wire/wireless network 300 through the communication unit 130.

In the present disclosure, the user terminal 200 may be paired with or connected to the device 100, in which the dementia identification model is stored, through the wire/wireless network 300, thereby transmitting/receiving and displaying predetermined data.

The user terminal 20 described in the present disclosure may include any device that transmits, receives, and displays at least one of data, content, service, and application. In addition, the user terminal 200 may be a terminal of a user who wants to check dementia. However, the present disclosure is not limited thereto.

In the present disclosure, the user terminal 200 may include, for example, a mobile device such as a mobile phone, a smart phone, a tablet PC, or an ultrabook. However, the present disclosure is not limited thereto, and the user terminal 200 may include a standing device such as a Personal Computer (PC), a microprocessor, a mainframe computer, a digital processor, or a device controller.

The user terminal 200 includes a processor 210, a storage 220, a communication unit 230, an image acquisition unit 240, a display 250, and a sound output unit 260. The components shown in FIG. 1 are not essential in implementing the user terminal 200, and thus, the user terminal 20 described in the present disclosure may have more or fewer components than those listed above.

Each component of the user terminal 200 of the present disclosure may be integrated, added, or omitted according to the specifications of the user terminal 200 that is actually implemented. That is, as needed, two or more components may be combined into one component, or one component may be subdivided into two or more components. In addition, the function performed in each block is for explaining an embodiment of the present disclosure, and the specific operation or device does not limit the scope of the present disclosure.

Since the processor 210, storage 220 and communication unit 230 of the user terminal 200 are the same components as the processor 210, storage 220 and communication unit 230 of the user terminal 200, a duplicate description will be omitted, and differences therebetween are mainly described below.

In the present disclosure, the processor 210 of the user terminal 200 may display a plurality of screens including a plurality of objects to identify whether a user has dementia. Here, the plural screens may respectively include the plurality of objects at different positions thereof.

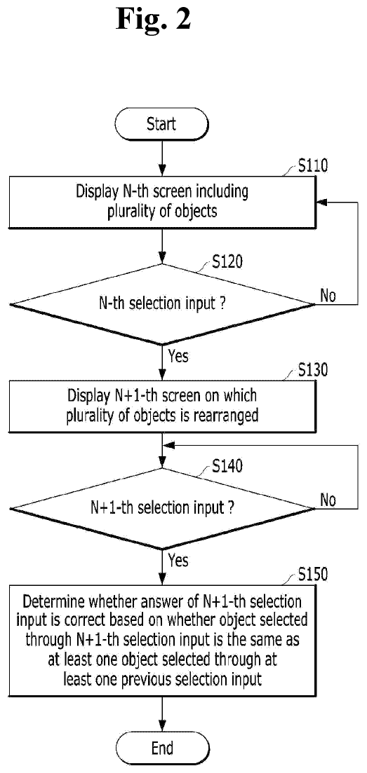

Specifically, the processor 210 may control the display 250 to display another screen in response to a selection input for selecting any one object among the plurality of objects included in the screen. Here, the N+1-th screen may be a screen on which a plurality of objects is rearranged. A detailed description thereof is described later with reference to FIG. 2.

Meanwhile, since high processing speed and computational power are required to perform an operation using the dementia identification model, the dementia identification model may be stored only in the storage 120 of the device 100 and may not be stored in the storage 220 of the user terminal 200. However, the present disclosure is not limited thereto.

The image acquisition unit 240 may include one or a plurality of cameras. That is, the user terminal 200 may be a device including one or plural cameras provided on at least one of a front part and rear part thereof.

The image acquisition unit 240 may process an image frame, such as a still image or a moving image, obtained by an image sensor. The processed image frame may be displayed on the display 250 or stored in the storage 220. Meanwhile, the image acquisition unit 240 provided in the user terminal 200 may match a plurality of cameras to form a matrix structure. A plurality of image information having various angles or focuses may be input to the user terminal 200 through the cameras forming the matrix structure as described above.

The image acquisition unit 240 of the present disclosure may include a plurality of lenses arranged along at least one line. The plurality of lenses may be arranged in a matrix form. The plural lenses may be arranged in a matrix form. Such cameras may be called an array camera. When the image acquisition unit 240 is configured as an array camera, images may be captured in various ways using the plural lenses, and images of better quality may be acquired.

According to some embodiments of the present disclosure, the image acquisition unit 240 may acquire an image including the user's eyes of the user terminal in association with display of a specific screen on the user terminal 200.

The display 250 may display (output) information processed by the user terminal 200. For example, the display 250 may display execution screen information of an application program driven in the user terminal 200, or User Interface (UI) and Graphic User Interface (GUI) information according to the execution screen information.

The display 250 may include at least one of a Liquid Crystal Display (LCD), a Thin-Film Transistor-Liquid Crystal Display (TFT LCD), an Organic Light-Emitting Diode (OLED), a flexible display, a 3(d) display, an e-ink display. However, the present disclosure is not limited thereto.

The display 250 may include a touch sensor for detecting a touch on the display 250 so as to receive a control command input by a touch method. The display 250 may implement a touch screen by forming a layered structure with the touch sensor or being integrally formed therewith. Such a touch screen may function as a user input part providing an input interface between the test device and the user and, at the same time, may provide an output interface between the test device and the user.

The touch sensor may detect a touch (or a touch input or a selection input) applied to the display 250 using at least one of various touch methods such as a resistive film method, a capacitive method, an infrared method, an ultrasonic method, and a magnetic field method.

For example, the touch sensor may be configured to convert a change in pressure applied to a specific part of the touch screen or a change in capacitance occurring in a specific part of the touch screen into an electrical input signal. The touch sensor may be configured to detect a position and area touched on the touch sensor by a touch object applying a touch on the touch screen, a pressure at the time of the touch, an capacitance at the time of the touch, etc.

Here, the touch object is an object that applies a touch to the touch sensor, and may be, for example, a finger, a touch pen, a stylus pen, a pointer, or the like.

As such, when there is a touch input (or selection input) to the touch sensor, a signal(s) corresponding thereto is sent to a touch controller. The touch controller processes the signal(s) and then sends corresponding data to the processor 210. Accordingly, the processor 210 may know which area of the display 250 has been touched, and the like. Here, the touch controller may be a component separate from the processor 210, or may be the processor 210 itself.

According to some embodiments of the present disclosure, the display 250 may display an N-th screen including a plurality of objects when the first task is performed. In addition, the display 250 may display the N+1-th screen in which a plurality of objects is rearranged when the second task is performed. Here, N may be a natural number of 1 or more. In addition, the N-th screen and the N+1-th screen may be screens in which the same plurality of objects is included in different positions.

Meanwhile, the user may perform a touch input to select any one of the plurality of objects in a state in which the N-th screen or the N+1-th screen is displayed. When a touch input to a target object is detected, the processor 210 may recognize that the target object is selected from among a plurality of objects.

The sound output unit 260 may output audio data (or sound data, etc.) received from the communication unit 230 or stored in the storage 220. The sound output unit 260 may also output a sound signal related to a function performed by the user terminal 200.

The sound output unit 260 may include a receiver, a speaker, a buzzer, and the like. That is, the sound output unit 260 may be implemented as a receiver or may be implemented in the form of a loudspeaker. However, the present disclosure is not limited thereto.

According to some embodiments of the present disclosure, the sound output unit 260 may output a preset sound (e.g., a voice describing what a user should perform through a first task or a second task) in connection with performing a first task or a second task. However, the present disclosure is not limited thereto.

According to some embodiments of the present disclosure, a specific screen may be displayed on the user terminal 200 to acquire input data input to the dementia identification model. This is described later in more detail with reference to FIG. 2.

FIG. 2 is a diagram for explaining an embodiment of a method of acquiring input data for dementia identification by a device according to some embodiments of the present disclosure. In describing FIG. 2, the contents overlapping with those described above in relation to FIG. 1 are not described again, and differences therebetween are mainly described below.

Referring to FIG. 2, the processor 110 of the device 100 may perform a first task S110 of causing an N-th screen including a plurality of objects to be displayed on the user terminal 200 (S110). Here, N may be a natural number of 1 or more.

For example, the processor 110 of the device 100 may generate an N-th screen including a plurality of objects to receive a first selection input for a first object among the plurality of objects and may transmit the generated N-th screen to the user terminal 200. In this case, the processor 210 of the user terminal 200 may control the display 250 to display the N-th screen including the plurality of objects.

As another example, a plurality of screens on which a plurality of objects is disposed at different positions may be stored in the storage 220 of the user terminal 200. When the processor 210 of the user terminal 200 receives a signal to display a screen including a plurality of objects from the device 100 through the communication unit 230, any one of the plurality of screens stored in the storage 220 may be displayed as an N-th screen.

As another example, images of a plurality of objects may be stored in the storage 220 of the user terminal 200. When the processor 210 of the user terminal 200 receives a signal to display a screen including the plurality of objects from the device through the communication unit 230, the processor 210 of the user terminal 200 may generate and display an N-th screen including the plurality of objects.

When the N-th screen is displayed in step S110, the processor 210 may check whether there is an N-th selection input for any one object (e.g., N-th object) among a plurality of objects (S120).

When the N-th selection input is not detected (S120, No), the display 250 of the user terminal 200 may continue to display the N-th screen including a plurality of objects.

When an N-th selection input for selecting any one of the plurality of objects is detected (S120, Yes), the processor 110 of the device 100 may perform a second task of causing the N+1-th screen, on which the plurality of objects is rearranged, to be displayed in the user terminal 200 (S130).

For example, when an N-th selection input is detected, the processor 210 of the user terminal 200 may control the communication unit 230 to transmit an N-th signal, which indicates that the N-th selection input is detected, to the device 100. Here, the N-th signal may include information on which object is selected from among the plurality of objects. When receiving the N-th signal, the processor 110 of the device 100 may control the communication unit 130 to generate an N+1-th screen on which the plurality of objects is rearranged and to transmit the generated N+1-th screen to the user terminal 200. When the N+1-th screen is received through the communication unit 230, the processor 210 of the user terminal 200 may control the display 250 to display the N+1-th screen.

As another embodiment, the storage 220 of the user terminal 200 may store a plurality of screens on which a plurality of objects is disposed at different positions. When an N-th selection input is detected, the user terminal 200 may transmit an N-th signal, which indicating that the N-th selection input is detected, to the device 100. Here, the N-th signal may include information on which object is selected from among the plurality of objects. In addition, the processor 210 may control the display 250 to select and display a previously undisplayed screen from among a plurality of screens stored in the storage 220. When a screen that has not been previously displayed is displayed, a screen on which a plurality of objects is rearranged may appear to be displayed.

As still another embodiment, images of a plurality of objects may be stored in the storage 220 of the user terminal 200. When the N-th selection input is detected, the user terminal 200 may transmit an N-th signal, which indicates that the N-th selection input is detected, to the device 100. Here, the N-th signal may include information on which object is selected from among the plurality of objects. The processor 210 of the user terminal 200 may generate an N+1-th screen on which a plurality of objects is rearranged so that the plurality of objects is displayed at different positions from the plurality of objects displayed on the N-th screen. In addition, the processor 210 may control the display 250 to display the N+1-th screen.

When the N+1-th screen is displayed in step S130, the processor 210 may check whether there is an N+1th selection input for any one of the plurality of objects (S140).

When the N+1-th selection input is not detected (S140, No), the display 250 of the user terminal 200 may continue to display the N+1-th screen on which the plurality of objects is rearranged.

When an N+1-th selection input for selecting any one of a plurality of objects displayed on the N+1-th screen is detected (S140, Yes), the processor 110 of the device 100 may perform a third task of determining whether the N+1-th selection input is correct based on whether an object selected through the N+1-th selection input is the same as the at least one object selected through at least one previous selection input (S150).

For example, when N is 1, the processor 110 may recognize whether an object selected through a second selection input is the same as an object selected through a first selection input that is a previous selection input. The processor 110 may determine that the answer is incorrect when the object selected through the first selection input is the same as the object selected through the second selection input, and may determine that the answer is correct when the object selected through the first selection input is different from the object selected through the second selection input.

As another embodiment, when N is 2, the processor 110 may recognize whether an object selected through a third selection input is the same as the plurality of objects selected through the previous first selection input and second selection input. The processor 110 may determine that the answer is correct when both the object selected through the first selection input and the object selected through the second selection input are different from the object selected through the third selection input, and may determine that the answer is incorrect when any one of the object selected through the first selection input and the object selected through the second selection input is the same as the object selected through the third selection input.

According to some embodiments of the present disclosure, the processor 110 of the device 100 may perform the second task and the third task a preset number of times. In this case, when the second task is performed M (a natural number greater than or equal to 1) more times, M may be added to N. That is, when the processor 110 detects a selection input for selecting any one of a plurality of objects included on the screen displayed on the user terminal 200, the processor 110 may cause the positions of the plurality of objects included in the screen displayed on the user terminal 200 to continuously change a preset number of times. In addition, the processor 110 may determine whether the current selection input is a correct answer based on whether all of the at least one object selected through the at least one previous selection input is the same as the object selected through the current selection input.

According to some embodiments of the present disclosure, the processor 110 may perform a preliminary task so that the user can check how to perform a task before performing the first task, the second task, and the third task. Here, since the preliminary task proceeds in the same manner as the first task, the second task, and the third task, a detailed description thereof is omitted.

The data acquired in the preliminary task may not be used as a digital biomarker that is input to the dementia identification model and used for identifying dementia. However, the present disclosure is not limited thereto the preliminary task, and data acquired in the preliminary task may also be used as a digital biomarker.

According to some embodiments of the present disclosure, the processor 110 of the device 100 may acquire gaze information based on an image including the user's eyes obtained in association with performing the first task and the second task. Here, the gaze information may include at least one of information on the order in which the user's gaze moves, information on whether the user's gaze is maintained on each of the plurality of objects displayed on the screen, and information on the time where the user's gaze is maintained on each of the plurality of objects.

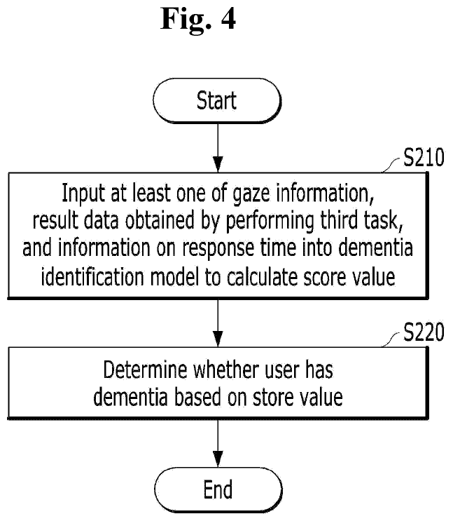

According to some embodiments of the present disclosure, the processor 110 may calculate a score value by inputting at least one of gaze information, result data obtained by performing the third task, and information on the user's response time into the dementia identification model. In addition, the processor 110 may determine whether a user has dementia based on the score value. This is described below in more detail with reference to FIG. 4.

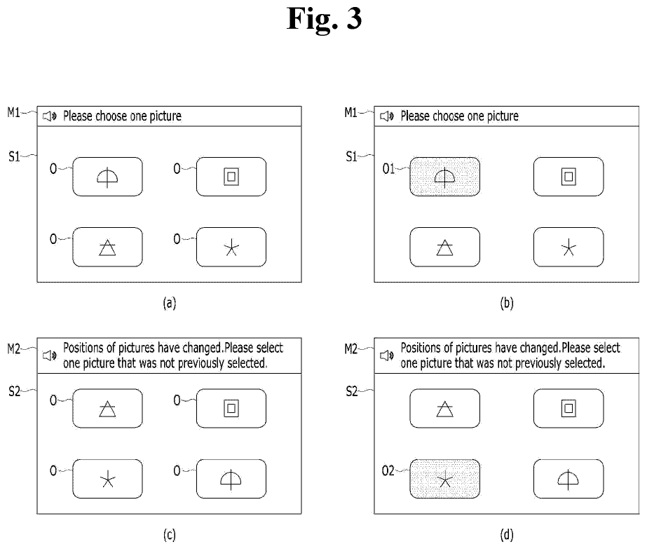

FIG. 3 is a diagram for explaining an embodiment of a screen displayed on a user terminal according to some embodiments of the present disclosure. In describing FIG. 3, the contents overlapping with those described above in relation to FIGS. 1 and 2 are not described again, differences therebetween are mainly described below.

Referring to FIG. 3(a), the user terminal 200 may control the display 250 to display an N-th screen S1 including a plurality of objects 0. Here, the plurality of objects 0 may be objects having at least one different shape and shape from each other.

Meanwhile, the N-th screen S1 may include a message M1 informing a user of a task to be performed through the currently displayed screen. For example, when the N-th screen S1 is a first screen that is first displayed, the message M1 may include a content to select any one of a plurality of objects included in the first screen. However, the present disclosure is not limited thereto.

According to some embodiments of the present disclosure, a sound related to the message M1 through the sound output unit 260 in conjunction with display of the message M1 (e.g., a voice explaining the content included in the message (M1) voice) may be output. In this way, when a sound is output together with the message M1 to allow the user to recognize a task to be performed by the user, it is possible to clearly understand what task the user should currently perform. Therefore, the possibility of performing a wrong operation by a simple mistake may be reduced.

Referring to FIG. 3(b), the processor 210 of the user terminal 200 may receive an N-th selection input for selecting one object 01 from among the plurality of objects 0.

In the present disclosure, when an N-th selection input is received, the processor 210 may inactivate an additional selection input for the N-th screen S1. That is, the second task may include a sub-task for inactivating the additional selection input for the N-th screen S1 when the N-th selection input is received. Here, the additional selection input may mean a selection input additionally detected after a selection input for first selecting any one object is detected in a state in which the N-th screen S1 is displayed.

As described above, when the additional selection input for the N-th screen S1 is inactivated, an error occurring when a user additionally touches an arbitrary area on the N-th screen S1 by mistake may be reduced.

Meanwhile, the processor 210 of the user terminal 200 may control the display 250 to reflect and display a preset effect on an object 01 selected from among the plurality of objects 0 through the N-th selection input. For example, only the object 01 selected from among the plurality of objects 0 through the N-th selection input may be highlighted in a color different from that of other objects. However, the present disclosure is not limited thereto. However, the present disclosure is not limited thereto.

Referring to FIG. 3(c), when an N-th selection input for selecting any one object 01 from among the plurality of objects in a state in which the N-th screen S1 of FIG. 3(b) is displayed is received, the processor 210 of the user terminal 200 may display a N+1-th screen S2 on which the plurality of objects is rearranged.

Referring to FIGS. 3(a) and (c), the respective positions of the plurality of objects 0 included in the N-th screen S1 may be different from the respective positions of the plurality of objects 0 included in the N+1-th screen.

That is, the second task of the present disclosure may include a sub-task of rearranging the plurality of objects included in the N+1-th screen by randomly changing the positions of the plurality of objects 0 included in the N-th screen S1.

For example, when an N-th selection input is detected, the processor 210 of the user terminal 200 may control the communication unit 230 to transmit an N-th signal, which indicates that the N-th selection input is detected, to the device 100. Here, the N-th signal may include information on which object 01 is selected from among a plurality of objects. When the processor 110 of the device 100 receives the N-th signal, the processor 110 of the device 100 randomly changes the positions of the plurality of objects 0 included in the N-th screen S1 to rearrange the plurality of objects included in the N+1-th screen S2. In addition, the processor 110 may control the communication unit 130 to transmit the N+1-th screen S2 to the user terminal 200. When the processor 210 of the user terminal 200 receives the N+1-th screen S2 through the communication unit 230, the processor 210 may control the display 250 to display the N+1-th screen S2.

As another example, images of a plurality of objects may be stored in the storage 220 of the user terminal 200. When an N-th selection input is detected, the user terminal 200 may transmit an N-th signal, which indicates that the N-th selection input is detected, to the device 100. Here, the N-th signal may include information on which object 01 is selected among the plurality of objects. The processor 210 of the user terminal 200 may randomly change the positions of the plurality of objects so that the plurality of objects is displayed at positions different from the plurality of objects displayed on the N-th screen S1 to generate the N+1-th screen S2. In addition, the processor 210 may control the display 250 to display the N+1-th screen S2.

Meanwhile, the N+1-th screen S2 may include a message M2 informing a user of a task to be performed through a currently displayed screen. For example, the message M2 may include a content to select any one object, not selected in the previous screen, from among the plurality of objects included in the N+1-th screen.

According to some embodiments of the present disclosure, a sound (e.g., a voice explaining the content included in the message M2) related to the message M2 through the sound output unit 260 may be output in association with display of the message M2. In this way, when outputting a sound together with the message M2 to let the user know what the user needs to do, it is possible to clearly understand what the user is currently doing. Therefore, the possibility of performing a wrong operation by a simple mistake may be reduced.

Referring to FIG. 3(d), the processor 210 of the user terminal 200 may receive an N+1-th selection input of selecting any one object 02 from among the plurality of objects 0 in a state in which the N+1-th screen S2 is displayed.

In the present disclosure, when the N+1-th selection input is received, the processor 210 may inactivate an additional selection input for the N+1-th screen S2. That is, the third task may include a sub-task that inactivates the additional selection input for the N+1-th screen S2 when the N+1-th selection input is received. Here, the additional selection input may refer to a selection input additionally detected after a selection input for first selecting any one object is detected in a state in which the N+1-th screen S2 is displayed.

As described above, when the additional selection input for the N+1-th screen S2 is inactivated, an error occurring when a user additionally touches an arbitrary area on the N+1-th screen S2 by mistake may be reduced.

Meanwhile, the processor 210 of the user terminal 200 may control the display 250 to reflect and display a preset effect on an object 01 selected from among the plurality of objects 0 through the N+1-th selection input. For example, only the object 01 selected from among the plurality of objects 0 through the N+1-th selection input may be highlighted in a color different from that of other objects. However, the present disclosure is not limited thereto.

Meanwhile, according to some embodiments of the present disclosure, when the processor 110 of the device 100 receives an N+1-th selection input of selecting any one 02 from among the plurality of objects 0 included in the N+1-th screen S2, the processor 110 may determine whether the answer of the N+1-th selection input is correct based on whether the object 01 selected through the N+1-th selection input is the same as at least one object selected from at least one previous selection input including the N-th selection input.

For example, when N is 1, the processor 110 of the device 100 may determine that the answer is incorrect when the object 02 selected through the second selection input is the same as the object 01 selected through the first selection input, and may determine that the second selection input is correct answer when the object 02 selected from the second selection input differs from the object 01 selected through the first selection input.

As another embodiment, when N is 2, the processor 110 of the device 100 may determine that the third selection input is a correct answer when an object 02 selected through the third selection input, an object 01 selected through the second selection input, and an object selected from the first selection input differ from each other, and may determine that the third selection input is an incorrect answer is incorrect when any one of an object 02 selected from the third selection input, an object 01 selected from the second selection input and an object selected from the first selection input is the same.

As a result, the third task may include an operation of determining that the answer is correct when all of the at least one object selected through the at least one previous selection input differs from the object selected from the N+1-th selection input; or an operation of determining that the answer is correct when any one of the at least one object selected from the at least one previous selection input is the same as the object selected from the N+1-th selection input.

Meanwhile, according to some embodiments of the present disclosure, information on whether the N+1-th selection input is correct may be utilized as a digital biomarker (a biomarker acquired through a digital device). This is described in more detail with reference to FIG. 4.

FIG. 4 is a flowchart for explaining an embodiment of a method of determining whether a user has dementia according to some embodiments of the present disclosure. In describing FIG. 4, the contents overlapping with those described above in relation to FIGS. 1 to 3 are not described again, and differences therebetween are mainly described below.

After performing the second task and the third task a preset number of times, the processor 110 of the device 100 may acquire at least one of gaze information, data as a result of performing the third task, and information on response time. Here, the gaze information, the result data, and the information on the response time may be digital biomarkers (biomarkers acquired through a digital device) for dementia identification.

The gaze information may be acquired by the device 100 or may be received by the device 100 after being acquired by the user terminal 200.

For example, the processor 210 of the user terminal 200 may acquire an image including the user's eyes through the image acquisition unit 240 while performing the first, second and third tasks. The processor 210 may control the communication unit 230 to directly transmit the image to the device 100. The processor 110 of the device 100 may receive the image through the communication unit 130. In this case, the processor 110 may acquire gaze information by analyzing the image.

As another embodiment, the processor 210 of the user terminal 200 may acquire an image including the user's eyes through the image acquisition unit 240 while performing the first, second and third tasks. The processor 210 may generate gaze information by analyzing the image. The processor 210 may control the communication unit 230 to transmit the first information to the device 100. In this case, the processor 110 may acquire gaze information by a method of receiving gaze information through the communication unit 130.

In the present disclosure, gaze information may be acquired by confirming the position of the user's pupil using only a B value among respective RGB values of a plurality of frames included in the image. Specifically, a region having a B value exceeding a preset threshold value in each of the plurality of frames may be recognized as a region in which the pupil is located. In this case, gaze information may be obtained based on a change in an area in which the pupil is located.

Meanwhile, according to some embodiments of the present disclosure, an image may be divided into the pupil and a background. A binarization process of changing a part corresponding to the pupil position to black and changing a part corresponding to the background to white may be applied to the image. After the binarization process, a flood fill may be applied to the image to remove noise from the image. Here, the flood fill may refer to an operation of replacing white pixels surrounded by black pixels with black pixels and replacing black pixels surrounded with white pixels with white pixels.

Meanwhile, in the present disclosure, the gaze information may include at least one of information on an order in which the user's gaze moves, information on whether the user's gaze is maintained on each of a plurality of objects, and information on the time where the user's gaze is maintained on each of the plurality of objects. However, when the gaze information includes all of the above-described information, the accuracy of dementia identification may be improved.

The information on the order in which the user's eyes move may refer to information on the order of gazing each of a plurality of objects included in an N-th screen (or an N+1-th screen) in a state in which the N-th screen (or the N+1-th screen) is displayed. However, the present disclosure is not limited thereto.

The information on whether the user's gaze is maintained with respect to each of the plurality of objects may refer to information on whether the user gazes at all of the plurality of objects once. However, the present disclosure is not limited thereto.

The information on the time where the user's gaze is maintained on each of the plurality of objects may refer to information about the time where the user gazes at each of the plurality of objects when the user gazes at all of the plurality of objects once. However, the present disclosure is not limited thereto.

Meanwhile, referring to FIG. 4, the processor 110 of the device 100 may input at least one of gaze information, result data obtained by performing the third task, and information on response time into the dementia identification model to calculate a score value (S210). However, to improve the accuracy of dementia identification of the dementia identification model, the processor 110 of the device 100 may input all of gaze information, result data obtained by performing the third task, and information on response time into the dementia identification model.

In the present disclosure, the gaze information, result data obtained by performing the third task, and information on response time which are input to the dementia identification model may be digital biomarkers having a high correlation coefficient with dementia identification among various types of digital biomarkers. Accordingly, when dementia identification is determined using gaze information, result data obtained by performing the third task, and information on response time, the accuracy of dementia identification may be improved.

In the present disclosure, the result data may include at least one of information on the number of times, determined to be a correct answer through the third task, among a preset number of times; and information on the number of times, determined to be an incorrect answer through the third task, among a preset number of times.

In the present disclosure, the information on response time may include information on a time taken until an N-th selection input or an N+1-th selection input is received in a state in which an N-th screen or an N+1-th screen is displayed. That is, the information on response time may include information on a time taken until an N-th selection input is received in a state in which an N-th screen is displayed; and information on a time taken until an N+1-th selection input is received in a state in which an N+1-th screen is displayed. However, the present disclosure is not limited thereto.

In the present disclosure, the dementia identification model may refer to an artificial intelligence model having a pre-trained neural network structure to calculate a score value when at least one of the gaze information, the result data obtained by performing the third task, and the information on response time is input. In addition, the score value may mean a value capable of recognizing whether dementia is present according to the size of the value.

According to some embodiments of the present disclosure, a pre-learned dementia identification model may be stored in the storage 120 of the device 100.

The dementia identification model may be trained by a method of updating the weight of a neural network by back propagating a difference value between label data labeled in learning data and prediction data output from the dementia identification model.

In the present disclosure, the learning data may be acquired by performing the first task, the second task, and the third task according to some embodiments of the present disclosure by a plurality of test users through their test devices. Here, the learning data may include at least one of gaze information, result data obtained by performing the third task, and information on user's response time.

In the present disclosure, the test users may include a user classified as a patient with mild cognitive impairment, a user classified as an Alzheimer's patient, a user classified as normal, and the like. However, the present disclosure is not limited thereto.

In the present disclosure, the test device may refer to a device where various test users perform tests when securing learning data. Here, the test device may be a mobile device such as a mobile phone, a smart phone, a tablet PC, an ultrabook, etc., similarly to the user terminal 200 used for dementia identification. However, the present disclosure is not limited thereto.

In the present disclosure, the label data may be a score value capable of recognizing whether a patient is normal, is an Alzheimer's patient, and a patient with mild cognitive impairment. However, the present disclosure is not limited thereto.

A dementia identification model may be composed of a set of interconnected computational units, which may generally be referred to as nodes. These nodes may also be referred to as neurons. The neural network may be configured to include at least one node. Nodes (or neurons) constituting the neural network may be interconnected by one or more links.

In the dementia identification model, one or more nodes connected through a link may relatively form a relationship between an input node and an output node. The concepts of an input node and an output node are relative, and any node in an output node relationship with respect to one node may be in an input node relationship in a relationship with another node, and vice versa. As described above, an input node-to-output node relationship may be created around a link. One output node may be connected to one input node through a link, and vice versa.

In the relation between the input node and the output node connected through one link, a value of data of the output node may be determined based on data that is input to the input node. Here, the link interconnecting the input node and the output node may have a weight. The weight may be variable, and may be changed by a user or an algorithm so as for the neural network to perform a desired function.

For example, when one or more input nodes are connected to one output node by each link, the output node may determine an output node value based on values that are input to input nodes connected to the output node and based on a weight set in a link corresponding to each input node.

As described above, in the dementia identification model, one or more nodes may be interconnected through one or more links to form an input node and output node relationship in the neural network. The characteristics of the dementia identification model may be determined according to the number of nodes and links in the dementia identification model, a correlation between nodes and links, and a weight value assigned to each of the links.

The dementia identification model may consist of a set of one or more nodes. A subset of nodes constituting the dementia identification model may constitute a layer. Some of the nodes constituting the dementia identification model may configure one layer based on distances from an initial input node. For example, a set of nodes having a distance of n from the initial input node may constitute n layers. The distance from the initial input node may be defined by the minimum number of links that should be traversed to reach the corresponding node from the initial input node. However, the definition of such a layer is arbitrary for the purpose of explanation, and the order of the layer in the dementia identification model may be defined in a different way from that described above. For example, a layer of nodes may be defined by a distance from a final output node.

The initial input node may refer to one or more nodes to which data (i.e., at least one of gaze information, result data obtained by performing the third task, and information on user's response time) is directly input without going through a link in a relationship with other nodes among nodes in the neural network. Alternatively, in a relationship between nodes based on a link in the dementia identification model, it may mean nodes that do not have other input nodes connected by a link. Similarly, the final output node may refer to one or more nodes that do not have an output node in relation to other nodes among nodes in the neural network. In addition, a hidden node may refer to nodes constituting the neural network other than the first input node and the last output node.

In the dementia identification model according to some embodiments of the present disclosure, the number of nodes in the input layer may be greater than the number of nodes in the output layer, and the neural network may have a form wherein the number of nodes decreases as it progresses from the input layer to the hidden layer. In addition, at least one of gaze information, result data obtained by performing the third task, and information on user's response time may be input to each node of the input layer. However, the present disclosure is not limited thereto. However, the present disclosure is not limited thereto.

According to some embodiments of the present disclosure, the dementia identification model may have a deep neural network structure.

A Deep Neural Network (DNN) may refer to a neural network including a plurality of hidden layers in addition to an input layer and an output layer. DNN may be used to identify the latent structures of data.

DNN may include convolutional neural networks (CNNs), Recurrent Neural Networks (RNNs), auto encoders, Generative Adversarial Networks (GANs), and a Restricted Boltzmann Machines (RBM), a Deep Belief Network (DBN), a Q network, a U network, a Siamese network, a Generative Adversarial Network (GAN), and the like. These DNNs are only provided as examples, and the present disclosure is not limited thereto.

The dementia identification model of the present disclosure may be learned in a supervised learning manner. However, the present disclosure is not limited thereto, and the dementia identification model may be learned in at least one manner of unsupervised learning, semi supervised learning, or reinforcement learning.

Learning of the dementia identification model may be a process of applying knowledge for performing an operation of identifying dementia by the dementia identification model to a neural network.

The dementia identification model may be trained in a way that minimizes errors in output. Learning of the dementia identification model is a process of repeatedly inputting learning data (test result data for learning) into the dementia identification model, calculating errors of an output (score value predicted through the neural network) and target (score value used as label data) of the dementia identification model on the learning data, and updating the weight of each node of the dementia identification model by backpropagating the error of the dementia identification model from an output layer of the dementia identification model to an input layer in a direction of reducing the error.

A change amount of a connection weight of each node to be updated may be determined according to a learning rate. Calculation of the dementia identification model on the input data and backpropagation of errors may constitute a learning cycle (epoch). The learning rate may be differently applied depending on the number of repetitions of a learning cycle of the dementia identification model. For example, in an early stage of learning the dementia identification model, a high learning rate may be used to enable the dementia identification model to quickly acquire a certain level of performance, thereby increasing efficiency, and, in a late stage of learning the dementia identification model, accuracy may be increased by using a low learning rate.

In the learning of the dementia identification model, the learning data may be a subset of actual data (i.e., data to be processed using the learned dementia identification model), and thus, there may be a learning cycle wherein errors for learning data decrease but errors for real data increase. Overfitting is a phenomenon wherein errors on actual data increase due to over-learning on learning data as described above.

Overfitting may act as a cause of increasing errors in a machine learning algorithm. To prevent such overfitting, methods such as increasing training data; regularization; and dropout that deactivate some of nodes in a network during a learning process, and utilization of a batch normalization layer may be applied.

Meanwhile, when a score value is acquired through step S220, the processor 110 may determine whether dementia is present based on the score value (S230).

Specifically, the processor 110 may determine whether dementia is present based on whether the score value exceeds a preset threshold value.

For example, the processor 110 may determine that a user has dementia when recognizing that the score value output from the dementia identification model exceeds the preset threshold value.

As another example, the processor 110 may determine that a user does not have dementia when recognizing that the score value output from the dementia identification model is less than or equal to the preset threshold value.

The above-described embodiments are only provided as examples, and the present disclosure is not limited to the embodiments.

According to some embodiments of the present disclosure, the processor 110 of the device 100 may acquire user identification information before performing the above-described first task, second task, and third task. Here, the user identification information may include user's age information, gender information, name, address information, and the like. In addition, at least a portion of the user identification information may be used as input data of the dementia identification model together with at least one of gaze information, result data obtained by performing the third task, and information on user's response time. Specifically, age information and gender information may be used as input data of the dementia identification model together with at least one of gaze information, result data obtained by performing the third task, and information on user's response time. In this way, when a score value is acquired after inputting at least a portion of the user identification information, together with at least one of gaze information, result data obtained by performing the third task, and information on user's response time, to the dementia identification model, the accuracy of dementia identification may be further improved. In this case, the dementia identification model may be a model wherein learning is completed based on at least a portion of the user identification information and at least one of gaze information, result data obtained by performing the third task, and information on user's response time.

120 people in a cognitive normal group and 9 people in a cognitively impaired group conducted an experiment to identify whether they had dementia through their user terminal. The goal of this experiment was to confirm the accuracy of the pre-learned dementia identification model. Specifically, the device 100 determined whether dementia was present based on a score value generated by inputting at least one of gaze information, result data obtained by performing the third task, and information on user's response time, which are acquired by performing the first task, the second task, and the third task, to the dementia identification model of the present disclosure. It was confirmed that the classification accuracy calculated through the above-described experiment was 80% or more.

According to at least one of the above-described several embodiments of the present disclosure, dementia may be accurately diagnosed in a method in which a patient hardly feels rejection.

In the present disclosure, the configurations and methods of the above-described several embodiments of the device 100 are not limitedly applied, and all or parts of each of the embodiments may be selectively combined to allow various modifications.

Various embodiments described in the present disclosure may be implemented in a computer or similar device-readable recording medium using, for example, software, hardware, or a combination thereof.

According to hardware implementation, some embodiments described herein may be implemented using at least one of Application Specific Integrated Circuits (ASICs), Digital Signal Processors (DSPs), Digital Signal Processing Devices (DSPDs), Programmable Logic Devices (PLDs), Field Programmable Gate Arrays (FPGAs), processors, controllers, micro-controllers, microprocessors, and other electrical units for performing functions. In some cases, some embodiments described in the present disclosure may be implemented with at least one processor.

According to software implementation, some embodiments such as the procedures and functions described in the present disclosure may be implemented as separate software modules. Each of the software modules may perform one or more functions, tasks, and operations described in the present disclosure. A software code may be implemented as a software application written in a suitable programming language. Here, the software code may be stored in the storage 120 and executed by at least one processor 110. That is, at least one program command may be stored in the storage 120, and the at least one program command may be executed by the at least one processor 110.

The method of identifying dementia by the at least one processor 110 of the device 100 using the dementia identification model according to some embodiments of the present disclosure may be implemented as code readable by the at least one processor in a recording medium readable by the at least one processor 110 provided in the device 100. The at least one processor-readable recording medium includes all types of recording devices in which data readable by the at least one processor 110 is stored. Examples of the at least one processor-readable recording medium includes Read Only Memory (ROM), Random Access Memory (RAM), CD-ROM, a magnetic tape, a floppy disk, an optical data storage device, and the like.

Meanwhile, although the present disclosure has been described with reference to the accompanying drawings, this is only an embodiment and the present disclosure is not limited to a specific embodiment. Various contents that can be modified by those of ordinary skill in the art to which the present disclosure belongs also belong to the scope of rights according to the claims. In addition, such modifications should not be understood separately from the technical spirit of the present disclosure.

1. A method of identifying dementia by at least one processor of a device, the method comprising:
performing a first task of causing a user terminal to display an N-th screen comprising a plurality of objects, wherein the N is a natural number equal to or greater than 1;
performing a second task of causing the user terminal to display an N+1-th screen wherein the plurality of objects is rearranged at positions on the N+1-th screen which are different from positions of the plurality of objects comprised in the N-th screen when an N-th selection input of selecting any one from among the plurality of objects comprised in the N-th screen is received; and
performing, when an N+1-th selection input for selecting any one from among the plurality of objects comprised in the N+1-th screen is received, a third task of determining whether an answer of the N+1-th selection input is correct based on whether the object selected from the N+1-th selection input is the same as at least one object selected from at least one previous selection input comprising the N-th selection input.

2. The method according to claim 1, further comprising: when the second task and the third task are performed a preset number of times and M is added to the N when the second task is performed the M more times, where the M is a natural number equal to or greater than 1.

3. The method according to claim 2, further comprising: acquiring gaze information based on an image comprising user's eyes acquired in association with performing the first task and the second task.

4. The method according to claim 3, wherein the gaze information comprises at least one of information about an order in which the user's gaze moves, information on whether the user's gaze is maintained on each of the plurality of objects, and information on a time where the user's gaze is maintained on each of the plurality of objects.

5. The method according to claim 3, comprising:
inputting at least one of the gaze information, result data obtained by performing the third task, and information on a response time of the user into a dementia identification model to calculate a score value; and
determining whether the user has dementia based on the score value.

6. The method according to claim 5, wherein the result data comprises at least one of information on the number of times determined to be a current answer through the third task among the preset number of times and information on the number of times determined to be an incorrect answer through the third task among the preset number of times, and
the information on the response time comprises information on a time taken until the N-th selection input or the N+1-th selection input is received in a state in which the N-th screen or the N+1-th screen is displayed.

7. The method according to claim 1, wherein the second task comprises a sub-task of inactivating an additional selection input for the N-th screen when the N-th selection input is received, and
the third task comprises a sub-task of inactivating an additional selection input for the N+1-th screen when the N+1-th selection input is received.

8. The method according to claim 1, wherein the third task comprises:
an operation of determining that an answer is correct when all of the at least one object selected through the at least one previous selection input differ from the object selected from the N+1-th selection input; or
an operation of determining that an answer is incorrect when any one of the at least one object selected from the at least one previous selection input is the same as the object selected from the N+1-th selection input.

9. The method according to claim 1, wherein the second task comprises a sub-task of randomly changing positions of the plurality of objects comprised in the N-th screen to rearrange the plurality of objects comprised in the N+1-th screen.

10. A computer program stored on a computer-readable storage medium, the computer program performs processes of identifying dementia when executed on at least one processor of a device, the processes comprising:
performing a first task of causing a user terminal to display an N-th screen comprising a plurality of objects, wherein the N is a natural number equal to or greater than 1;
performing a second task of causing the user terminal to display an N+1-th screen wherein the plurality of objects is rearranged at positions on the N+1-th screen which are different from positions of the plurality of objects comprised in the N-th screen when an N-th selection input of selecting any one from among the plurality of objects comprised in the N-th screen is received; and
performing, when an N+1-th selection input for selecting any one from among the plurality of objects comprised in the N+1-th screen is received, a third task of determining whether an answer of the N+1-th selection input is correct based on whether the object selected from the N+1-th selection input is the same as at least one object selected from at least one previous selection input comprising the N-th selection input.

11. A device for identifying dementia, the device comprises:
a storage configured to store at least one program command; and
at least one processor configured to perform the at least one program command,
wherein the at least one processor performs a first task of causing a user terminal to display an N-th screen comprising a plurality of objects, wherein the N is a natural number equal to or greater than 1;
performs a second task of causing the user terminal to display an N+1-th screen wherein the plurality of objects is rearranged at positions on the N+1-th screen which are different from positions of the plurality of objects comprised in the N-th screen when an N-th selection input of selecting any one from among the plurality of objects comprised in the N-th screen is received; and
performs, when an N+1-th selection input for selecting any one from among the plurality of objects comprised in the N+1-th screen is received, a third task of determining whether an answer of the N+1-th selection input is correct based on whether the object selected from the N+1-th selection input is the same as at least one object selected from at least one previous selection input comprising the N-th selection input.
